(19) United States
(12) Patent Application Publication
(10) Pub. No.: US 2015/0366498 A1
(43) Pub. Date: Dec. 24, 2015

(54) PORTABLE APPARATUS FOR MONITORING URINARY BLADDER INTERNAL PRESSURE

(71) Applicant: SOGANG UNIVERSITY RESEARCH FOUNDATION, Seoul (KR)

(72) Inventors: Bum-Kyoo CHOI, Seoul (KR); Ho-Young LEE, Yongin-si (KR); Jeong-Ho EOM, Seoul (KR)

(73) Assignee: SOGANG UNIVERSITY RESEARCH FOUNDATION, Seoul (KR)

(21) Appl. No.: 14/758,771

(22) PCT Filed: Dec. 23, 2013
(86) PCT No.: PCT/KR2013/012048

§ 371 Date: Jun. 30, 2015

(30) Foreign Application Priority Data
Dec. 31, 2012 (KR) 10-2012-0158163

(51) Int. Cl. A61B 5/20; A61B 5/00; A61M 25/00

(52) U.S. Cl. CPC A61B 5/205; A61M 25/0067; A61B 5/6855; A61B 5/742; A61M 2025/0002

(57) ABSTRACT

A portable apparatus for monitoring urinary bladder internal pressure includes: a urinary catheter being provided with a pressure measuring sensor and a fixing member for dwelling the pressure measuring sensor in a bladder and being inserted into the bladder to discharge urine; and a display device which is connected to the pressure measuring sensor and displays a measured signal measured by the pressure measuring sensor. Since a urinary catheter having a fixing member on which a pressure measuring sensor of a thin-film-type is attached is inserted into a patient and at the same time the pressure of the bladder is monitored in real time by connecting the pressure measuring sensor to a portable display device, urination according to the pressure information of the bladder can be performed in a precise timing.

### FIELD OF INVENTION

The present invention relates to a portable apparatus for monitoring internal pressure of a bladder.

### BACKGROUND ART

Generally, in order to diagnose the state of a patient who has dysuresia such as micturition disorder or urinary incontinence or neurogenic vesical dysfunction(neurogenic bladder), it is required to measure pressure change in a state of fullness of urine and during urination and to check the function of the bladder.

A method of filling physiological saline in the bladder of a patient instead of urine until a patient feels the desire to micturition and then making a patient urinate and measuring internal pressure of the bladder has been used.

However, in this method, instead of waiting until the bladder of a patient is fully filled with urine, the bladder is artificially filled with physiological saline, so there is a limit in reproducing physiological phenomenon.

Accordingly, in order to solve this problem, a method in which after a urethra catheter which is inserted into the urethra for measuring the pressure and an external measuring equipment to the body of a patient, when a patient feels the desire to micturition, a patient urinates in a state that the catheter is dwelled in the body, and the bladder internal pressure is measured has been introduced.

However, in this method, a patient must go to the hospital to test, and this makes a patient inconvenient and uncomfortable.

In addition, since the test for measuring the internal pressure of the bladder is performed in a specific test room of a hospital, a patient feels psychological burdens, and this makes it difficult to reproduce the natural physiological phenomenon and thus the accuracy of the test becomes deteriorated.

### DETAILED DESCRIPTION OF THE INVENTION

### Technical Problem

The present invention has been made in an effort to provide a portable apparatus for monitoring internal pressure of a bladder in which a physiological phenomenon is reproduced using a urinary catheter which is dwelled in a body of a patient who suffers micturition disorder to urinate by oneself, and at the same time a urination timing and a current state of a patient are informed to the patient by the monitoring of a display device so as to reduce inconvenience in a daily life.

### Technical Solution

A portable apparatus for monitoring urinary bladder internal pressure according to an exemplary embodiment of the present invention includes: a urinary catheter being provided with a pressure measuring sensor and a fixing member for dwelling the pressure measuring sensor in a bladder and being inserted into the bladder to discharge urine; and a display device which is connected to the pressure measuring sensor and displays a measured signal measured by the pressure measuring sensor.

The fixing member may be made of material which can be expanded and contracted and is fixed within the bladder on expanding by a predetermined length.

The predetermined length may be greater than a width of a urethra into which the urinary catheter is inserted.

The urinary catheter may include an inserted portion which is inserted into a body and dwelled therein and an exposed portion which is connected to the inserted portion and is exposed to the outside of the body, the inserted portion may include a urine inlet into which the urine in the bladder flows and a fluid inlet into which fluid for expanding the fixing member flows, and the exposed portion may include an outlet port for discharging the urine flowing into the urine inlet and a control portion for controlling the fluid and the pressure measuring sensor.

The urinary catheter may include a first fluid channel which is connected to the outlet port from the urine inlet to allow the urine flow therethrough and a second flow channel which is connected to the control portion from the fluid inlet to allow the fluid flow therethrough.

The pressure measuring sensor may be formed a thin-film-type electrode pattern which is able to be attached to a surface of the fixing member to undergo change in a shape depending on expansion and contraction of the fixing member and generates a corresponding signal.

The pressure measuring sensor may be an electrical-resistance-type electrode pattern using resistance change depending on change in length due to internal pressure of the bladder.

The pressure measuring sensor may be a capacitance-type electrode pattern which detects change in capacitance due to change in a distance between two electrodes as a signal.

The display device may be connected to the pressure measuring sensor via one of a wire connection and a wireless connection.

The pressure measuring sensor may be connected to an external device via a wire which is inserted into the urinary catheter for supplying of electricity and for detecting a signal due to internal pressure of the bladder.

The wire may be disposed along a fluid channel formed within the urinary catheter or is inserted into an outer wall of the urinary catheter by molding to be connected to the outside.

### Advantages Effects

According to the present invention, since a urinary catheter having a fixing member on which a pressure measuring sensor of a thin-film-type is attached is inserted into a patient and at the same time the pressure of the bladder is monitored in real time by connecting the pressure measuring sensor to a portable display device, urination according to the pressure information of the bladder can be performed in a precise timing.

Further, since a patient can check the pressure information of his bladder via the portable display device and can urinate, a patient needs not to visit a hospital to checking so that inconvenience and cost for test can be reduced, and the reproducing of the physiological phenomenon in a daily life and the real time monitoring are possible so that the state of the patient can be precisely diagnosed.

### BRIEF DESCRIPTION OF DRAWINGS

FIG. 1 is a conceptual diagram of a portable apparatus for monitoring urinary bladder internal pressure according to an embodiment of the present invention.

FIG. 2 is an enlarged view of a portion “A” in FIG. 1.

FIG. 3 is a reference drawing showing states in which a pressure measuring sensor of a portable apparatus for monitoring urinary bladder internal pressure according to an embodiment of the present invention is indwelled in a bladder.

FIG. 4 is a sectional view taken along a line IV-IV in FIG. 2.

FIG. 5 is a reference drawing showing an electrode pattern of an electric-resistance type pressure measuring sensor of a portable apparatus for monitoring urinary bladder internal pressure according to an embodiment of the present invention.

FIG. 6 is a reference drawing showing an electrode pattern of a capacitance type pressure measuring sensor of a portable apparatus for monitoring urinary bladder internal pressure according to an embodiment of the present invention.

FIG. 7 is a reference drawing showing a case in which a plurality of pressure measuring sensors of a portable apparatus for monitoring urinary bladder internal pressure according to an embodiment of the present invention are provided in a urinary catheter.

### DETAILED DESCRIPTION OF THE EMBODIMENTS

Embodiments of the present invention will be explained in detail hereinafter with reference to the accompanying drawings.

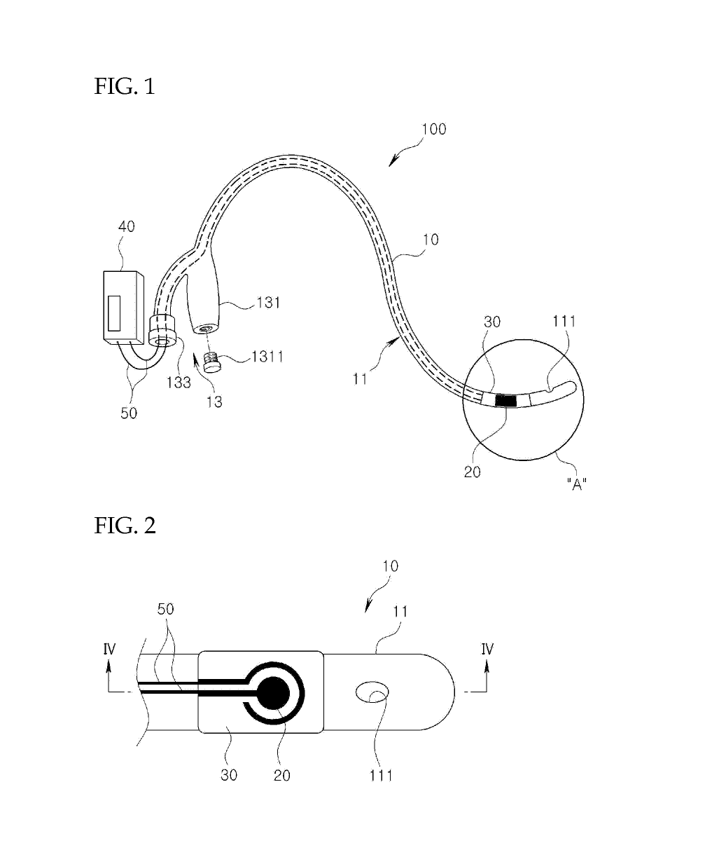

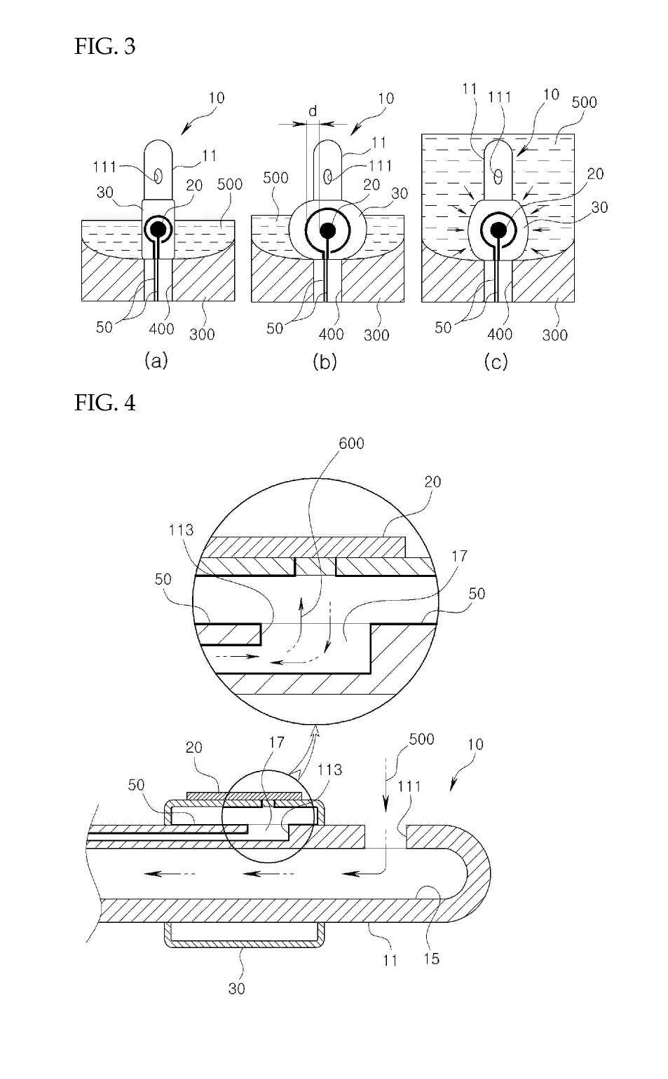

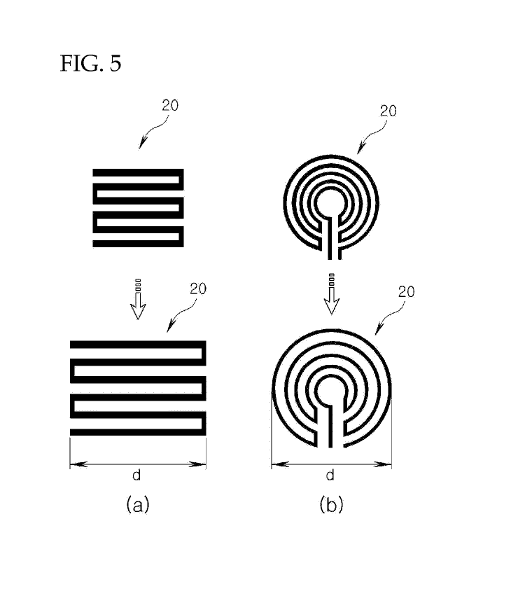

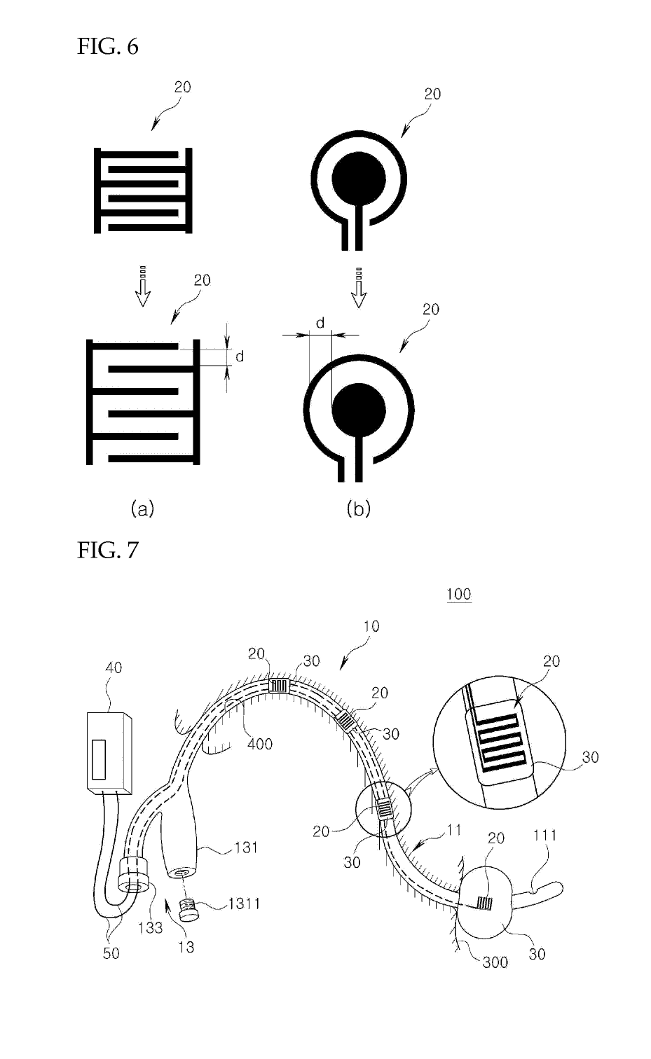

FIG. 1 is a conceptual diagram of a portable apparatus for monitoring urinary bladder internal pressure according to an embodiment of the present invention, FIG. 2 is an enlarged view of a portion “A” in FIG. 1, FIG. 3 is a reference drawing showing states in which a pressure measuring sensor of a portable apparatus for monitoring urinary bladder internal pressure according to an embodiment of the present invention is indwelled in a bladder, FIG. 4 is a sectional view taken along a line IV-IV in FIG. 2, FIG. 5 is a reference drawing showing an electrode pattern of an electric-resistance type pressure measuring sensor of a portable apparatus for monitoring urinary bladder internal pressure according to an embodiment of the present invention, FIG. 6 is a reference drawing showing an electrode pattern of a capacitance type pressure measuring sensor of a portable apparatus for monitoring urinary bladder internal pressure according to an embodiment of the present invention, and FIG. 7 is a reference drawing showing a case in which a plurality of pressure measuring sensors of a portable apparatus for monitoring urinary bladder internal pressure according to an embodiment of the present invention are provided in a urinary catheter.

Referring to FIG. 1 and FIG. 2, a portable apparatus 100 for monitoring urinary bladder internal pressure according to an embodiment of the present invention (hereinafter referred to as portable apparatus 100 for monitoring urinary bladder internal pressure) includes a urinary catheter 10 and a display device 40.

First, the urinary catheter 10 will be described.

The urinary catheter 10 is formed in a tube shape, and is generally formed to include an inserted portion 11 which is inserted into a body to be indwelled within a urethra 400 and a bladder 300, and an exposed portion which is connected to the inserted portion 11 and is exposed to the outside.

In more detail, the urinary catheter 10 is provided with a pressure measuring sensor 20 outside the inserted portion 11 and a fixing member 30 which indwells the pressure measuring sensor 20 within a bladder 30, and is inserted into the bladder 300 to discharge urine within the bladder 300 to the outside.

Here, the pressure measuring sensor 20 is provided to a head portion of the urinary catheter 10 which passes through the urethra 400 and is protruded toward the inside of the bladder 300 and measures the pressure inside the bladder 300, and at this time a fixing member 30 which can fix the urinary catheter 10 inside the bladder 300 may be provided to the head portion of the urinary catheter 10 which is inserted into the bladder 300 to be dwelled therein.

In more detail, referring to FIG. 3, the urinary catheter 10 includes the fixing member 30 which can fix the urinary catheter 10 which is provided with the pressure measuring sensor such that the pressure measuring sensor 20 is dwelled inside the bladder 300, and the fixing member 30 may be formed of material which can be expanding and contracting. That is, the fixing member 30 is provided at an outer surface of the urinary catheter 10 and is formed of material which can be expanding and contacting according to whether there is pressure from the outside or not, so it can fix the urinary catheter 10 inside the bladder 300 with expansion thereof. As an example, the fixing member 30 may be a balloon which is made of elastic material which can be expanding when fluid enters therein. However, the fixing member 30 may be formed of various material and may have various shape according to the necessity in use.

At this time, the fixing member 30 may be formed so as to be fixed inside the bladder 300 when it expands by a predetermined length within the ladder 300. In more detail, the fixing member 30 may be fixed inside the bladder 300 when it expands greater than the width of the urethra 400 into which the urinary catheter 10 is inserted. For example, the fixing member 30 is positioned at the head portion of the urinary catheter 10 and expands greater than the urethra 400 by external pressure, so that it is caught by a neck portion connecting the urethra 400 and the bladder 30 so as not to be dropped out toward the urethra 400 and to be fixed inside the bladder 300. That is, the fixing member 30 expands so that that the urinary catheter 10 is fixed inside the bladder 300, and accordingly the pressure measuring sensor 20 may also be dwelled inside the bladder 300.

In addition, the inserted portion 11 of the urinary catheter 10 has two inlets.

Referring to FIG. 1 and FIG. 4, the inserted portion 11 of the urinary catheter 10 may include a urine inlet 111 into which urine 500 inside the bladder 300 flows and a fluid inlet 113 into which a fluid 600 which is able to expand the fixing member 300 flows. In more detail, the urine inlet 111 into which the urine 500 within the bladder 300 flows is formed at one side of the inserted portion 11 and the fluid inlet 113 which communicates with the fixing member 30 which is provided to the outer side of the inserted portion 11 and through which the fluid 600 which is able to expand the fixing member 30 inflows or outflows.

Accordingly, the inlets which are composed of the urine inlet 111 and the fluid inlet 113 are provided to the inserted portion 11 of the urinary catheter 10, and through these, the urine 500 can be discharged and the fixing member 30 can be expanded.

The exposed portion 13 of the urinary catheter 10 has two ports.

Referring to FIG. 1, the exposed portion of the urinary catheter 10 may include an outlet port 131 for discharging urine which is introduced through the urine inlet 111 and a control port 133 for control of the fluid and the pressure measuring sensor 20. In more detail, the outlet port 131 and the control port 133 of the exposed portion 13 may be formed to be connected with the inserted portion 11 separately, and the control port 13 may be additionally formed to one side of the outlet port 131 which is extended from the inserted portion 11. Here, the control port 133 may be directly or indirectly connected to an outer supplying device which can supply fluid which flows into the fixing member 30. That is, the fluid supply to the fixing member 30 is performed through the control port 133, so expansion or contraction of the fluid can be controlled. In addition, the control port 133 may play a role of a passage through which a wire 50 which is connected to the pressure measuring sensor 20 can be connected to the outer device in order to transmit measured signals of the pressure measuring sensor 20 to the outer device passes. Accordingly, the control port 133 may be provided to the exposed portion 13 for the control of the fluid which flows into the fixing member 30 and the control of the pressure measuring sensor 20.

Here, a stopper 1311 which can regulate the discharging of urine may be provided to the outlet port 131 which is formed to the exposed portion 13 of the urinary catheter 10. Exemplarily, the stopper 1311 may be a passive type or an active type depending on the using state of a user, the passive type stopper is a type in which a user can open or close by hand, and the active type stopper is a type in which opening and closing are performed by external electrical or magnetic signal. Accordingly, the active type stopper can protect the secondary infection which may be caused by the patient's operation, when compared to the passive type stopper. The portable motoring device 100 for internal pressure of bladder may have an advantage in using the active type stopper. However, the stopper 1311 can be variously varied depending on the necessity in use.

Meanwhile, the urinary catheter 10 may form two flow channels which are formed by the connection between the urine inlet 111 and the fluid inlet 113 and the outlet port 131 and the control port 133.

Referring to FIG. 1 and FIG. 4, the urinary catheter 10 may have a first flow channel 15 which is connected from the urine inlet 111 to the outlet port 131 so that the urine 500 flows therein and a second flow channel 17 which is connected to the fluid inlet 113 to the control port 133 so that the fluid 600 flows therein.

In more detail, referring to the shape of the section of the urinary catheter 10, the urine inlet 111 which is formed to the urinary catheter 10 communicates with the inside of the urinary catheter 10 to which the first flow channel 15 is formed and the first flow channel 15 is connected to the outlet port 131, so the urine inlet 111 and the outlet port 131 are connected to one another via the first flow channel 15, and thus the urine 500 from the urine inlet 111 can be discharged to the outside through the outlet port 131.

In addition, the fluid inlet 113 which is formed in the urinary catheter 10 communicates with the inside of the fixing member 30 which is provided to the outside of the urinary catheter 10, and the second flow channel 17 is connected to the control port 133 in a state of being spaced from the first flow channel 15, so the fluid inlet 113 and the control port 133 are connected to one another via the second flow channel 17, so the fluid 600 from the external fluid supplying device which is connected to the control port 133 can be supplied to the fixing member 30 via the fluid inlet 113 so that the fixing member 30 can be controlled.

Referring to FIG. 5 and FIG. 6, the pressure measuring sensor 20 of the portable apparatus 100 for monitoring urinary bladder internal pressure may be formed as an electrode pattern of a thin film type which is attached to the outer surface of the fixing member 30 so as to be deformed depending on expansion and contraction of the fixing member 30 and generates the corresponding signal. That is, the pressure measuring sensor 20 has a contractility like the fixing member 30, and is attached to the outer surface of the fixing member 30 so as to undergo the change of its shape depending on the expansion and the contraction of the fixing member 30 and generate the signal for the changed state. For example, the electrode which is used for the electrode pattern may be formed of transparent conductive polymer or metal, but it may be altered depending on the necessity in use.

In more detail, the pressure measuring sensor 20 may be formed as one of the electrical-resistance-type electrode pattern and a capacitive-type electrode pattern and may be attached to the fixing member 30.

Referring to FIG. 5, the electrical-resistance-type electrode pattern may detect the internal pressure of the bladder 300 by obtaining change in resistance due to change in the length of the electrode by the internal pressure of the bladder 300. In more detail, the electrical-resistance-type electrode pattern may be formed such that a starting point and an ending point of the electrode pattern are connected by a single line without being disconnected. Accordingly, the total length of the electrical-resistance-type electrode pattern which is attached to the outer surface of the fixing member 30 is enlarged during the expansion of the fixing member 30, so the shape thereof may be changed from the initial shape. That is, the electrical-resistance-type electrode pattern may measure the pressure inside the bladder 300 based on the change in the length of the elongated electrode due to the expansion and the contraction.

Further, referring to FIG. 6, the capacitive-type electrode pattern may detect the internal pressure of the bladder 300 by detecting a signal of the change in the capacitance due to the change in the interval between both electrodes. In more detail, the capacitive-type electrode pattern may be formed such that the positive electrode and the negative electrode are separated from one another. Accordingly, the capacitive-type electrode pattern which is attached to the outer surface of the fixing member 30 undergoes the change in length and the increase of the gap between the patterns of the positive electrode and the negative electrode while the fixing member 30 expands, so the shape thereof becomes different from the initial state. That is, the capacitive-type electrode pattern may measure the pressure inside the bladder 300 based on the change in the interval between the patterns due to the expansion and the contraction of the fixing member 30.

Next, the display device 40 will be explained.

The display device 40 of the potable monitoring device 100 for internal pressure of a bladder is a portable device controlling the fluid 600 flowing into the fixing member 30 and the pressure measuring sensor 20 which can measure the internal pressure in the bladder 300 using communication function, and is connected to the pressure measuring sensor 20 to receive the measured signal from the pressure measuring sensor 20 and displays signals relating to the internal pressure of the bladder 300 on a display window.

In more detail, the display device 40 may be a portable terminal device which is connected to the pressure measuring sensor 20 via wire or wireless connection to control the same.

First, in case the display device 40 is connected to the pressure measuring sensor 20 by a wire connection, the pressure measuring sensor 20 may be connected to the display device 40 via a wire 50 which is inserted into the urinary catheter 10 for supplying electricity and for measuring a signal depending on the internal pressure of the bladder 300. Referring to FIG. 1 and FIG. 4, the wire 50 may be disposed along a flow channel within the urinary catheter 10 or may be disposed at the outside by being molded on the outer wall of the urinary catheter 10.

That is, the wire 50 is connected to the pressure measuring sensor 20 which is attached to the fixing member 30 and is disposed in the second flow channel 17 of the urinary catheter 10, and is extracted to the outside through the control port 133 to be connected to the display device 40, or is inserted into the outer wall of the urinary catheter 10 by molding to be extracted to the outside to be connected to the display device 40.

To the contrary, in case that the display device 40 is connected to the pressure measuring sensor 20 via the wireless connection, the pressure measuring sensor 20 is connected to a transmitter which is provided to the exposed portion 13 for supplying electricity and measuring a signal depending on the internal pressure of the bladder 300 via the wire, and the display device 40 can cooperate with the transmitter so monitor the change of the internal pressure of the bladder 300.

For example, in order to realize the display device 40 of the portable monitoring device 100 for internal pressure of a bladder, a dummy sensor (gauge) is disposed at the exposed portion 13 of the urinary catheter 10 or at a position free from the influence of the pressure and is circuitedly connected to the display device 40, and thus the signal process can be performed in a state of being minimally effected by the change in temperature or by the external noise signal. In more detail, after connecting the dummy sensor and the pressure measuring sensor 20 together to form a bridge circuit for measuring the respective electrode resistances, amplifying the voltage output from the bridge circuit using an amplifier and filtering the noise of the amplified voltage using a low-pass-filter, and the output value of the pressure measuring sensor 20 is displayed on the display device 40, so the internal pressure of the bladder 300 can be measured.

Meanwhile, the portable apparatus 100 for monitoring urinary bladder internal pressure may also be used for measuring the pressure in the urethra 400 into which the urinary catheter 10 is inserted as well as the internal pressure of the bladder 300.

Referring to FIG. 7, the portable apparatus 100 for monitoring urinary bladder internal pressure may be formed by attaching a plurality of fixing members 30 and a plurality of pressure measuring sensors 20 with predetermined intervals at the outer side of the urinary catheter 10 which is provided inside the urethra 400 so as to measure the pressure in the urethra 400 as well as the internal pressure of the bladder 300 at the same time.

The functions and the effects between the above-described components will be explained hereinafter.

Referring to FIG. 3, in case that the urinary catheter 10 is inserted into the bladder 300, the fluid is supplied to the fixing member 300 from the display device 40 for fixing the urinary catheter 10. The fixing member 30 is expanded by the supplied fluid to be fixed within the bladder 300, and thus the pressure measuring sensor 20 is elongated to undergo the change in shape. At this time, since the bladder 300 is gradually filled with the urine 500 as time passes, a pressure acts inwardly on the surface of the enlarged fixing member 30 so that the fixing member 30 contracts. In addition, the elongated pressure measuring sensor 20 which is attached to the outer surface of the fixing member 30 also gradually contracts due to the contraction of the fixing member 30. That is, the initial signal value of the pressure measuring sensor 20 is generated by the fixing member 30 which is expanded for fixing the inserted portion 11 of the urinary catheter 10, and the magnitude of the signal of the pressure measuring sensor 20 varies depending on the gradual increase of the urine 500 in the bladder 300.

Accordingly, the bladder pressure due to the filling level of the urine 500 in the bladder 300 is calculated from the calculation of the difference between these signals, and a user may see the signals which are generated at the time of expansion and contraction of the pressure measuring sensor 20 on the display device 40 connected to the pressure measuring sensor 20.

As described above, according to the portable monitoring device 100 for internal pressure of a bladder, since the urinary catheter 10 having the fixing member 30 on which the thin-film-type pressure measuring sensor 20 is attached is inserted to a patient, and at the same time the pressure measuring sensor is connected to the portable display device 40, the internal pressure of the bladder 300 can be monitored in real time, and thus self-catheterization can be performed at a precise timing.

Further, since the self-catheterization can be performed after recognizing the pressure information with the bladder from the portable display device 40, the continuity of life of the patient can be maintained. In addition, it is not required to visit a hospital for checking the urination disease to reduce inconvenience and cost, and the state of the patient can be exactly diagnosed by the reproducing and the real-time monitoring of the physiological phenomena.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

### INDUSTRIAL APPLICABILITY

The present invention relates to a portable monitoring device for internal pressure of a bladder which can be applied to medical devices, so it has an industrial applicability.

1. A portable apparatus for monitoring urinary bladder internal pressure comprising:
a urinary catheter being provided with a pressure measuring sensor and a fixing member for dwelling the pressure measuring sensor in a bladder and being inserted into the bladder to discharge urine; and
a display device which is connected to the pressure measuring sensor and displays a measured signal measured by the pressure measuring sensor.

2. The portable apparatus for monitoring urinary bladder internal pressure of claim 1, wherein the fixing member is made of material which can be expanded and contracted and is fixed within the bladder on expanding by a predetermined length.

3. The portable apparatus for monitoring urinary bladder internal pressure of claim 2, wherein the predetermined length is greater than a width of a urethra into which the urinary catheter is inserted.

4. The portable apparatus for monitoring urinary bladder internal pressure of claim 1, wherein the urinary catheter comprises an inserted portion which is inserted into a body and dwelled therein and an exposed portion which is connected to the inserted portion and is exposed to the outside of the body,
wherein the inserted portion comprises a urine inlet into which the urine in the bladder flows and a fluid inlet into which fluid for expanding the fixing member flows, and
wherein the exposed portion comprises an outlet port for discharging the urine flowing into the urine inlet and a control portion for controlling the fluid and the pressure measuring sensor.

5. The portable apparatus for monitoring urinary bladder internal pressure of claim 4, wherein the urinary catheter comprises a first fluid channel which is connected to the outlet port from the urine inlet to allow the urine flow therethrough and a second flow channel which is connected to the control portion from the fluid inlet to allow the fluid flow therethrough.

6. The portable apparatus for monitoring urinary bladder internal pressure of claim 1, wherein the pressure measuring sensor is formed a thin-film-type electrode pattern which is able to be attached to a surface of the fixing member to undergo change in a shape depending on expansion and contraction of the fixing member and generates a corresponding signal.

7. The portable apparatus for monitoring urinary bladder internal pressure of claim 1, wherein the pressure measuring sensor is an electrical-resistance-type electrode pattern using resistance change depending on change in length due to internal pressure of the bladder.

8. The portable apparatus for monitoring urinary bladder internal pressure of claim 1, wherein the pressure measuring sensor is a capacitance-type electrode pattern which detects change in capacitance due to change in a distance between two electrodes as a signal.

9. The portable apparatus for monitoring urinary bladder internal pressure of claim 1, wherein the display device is connected to the pressure measuring sensor via one of a wire connection and a wireless connection.

10. The portable apparatus for monitoring urinary bladder internal pressure of claim 1, wherein the pressure measuring sensor is connected to an external device via a wire which is inserted into the urinary catheter for supplying of electricity and for detecting a signal due to internal pressure of the bladder.

11. The portable apparatus for monitoring urinary bladder internal pressure of claim 10, wherein the wire is disposed along a fluid channel formed within the urinary catheter or is inserted into an outer wall of the urinary catheter by molding to be connected to the outside.
